(19) United States
(12) Patent Application Publication
(10) Pub. No.: US 2003/0199747 A1
(43) Pub. Date: Oct. 23, 2003

(54) Methods and apparatus for the identification and stabilization of vulnerable plaque

(76) Inventors: Kenneth J. Michlitsch, La Jolla, CA (US); Eduardo Ignacio Cespedes, Folsom, CA (US)

Correspondence Address: Nicola A. Pisano, Esq., Luce, Forward, Hamilton & Scripps LLP, 11988 El Camino Real, Ste 200, San Diego, CA, 92130, US

(21) Appl. No.: 10/127,052

(22) Filed: Apr. 19, 2002

(51) Int. Cl. A61B 6/00; A61B 8/00

(52) U.S. Cl. 600/407; 600/474; 600/437

(57) ABSTRACT

The present invention provides methods and apparatus for identifying and stabilizing vulnerable plaque via multi-functional catheters having both thermography and imaging capabilities. It is expected that correlating imaging and thermography data will facilitate improved identification of vulnerable plaque. Apparatus of the present invention may also be provided with optional stabilization elements for stabilizing vulnerable plaque. Methods of using apparatus of the present invention are provided.

### FIELD OF THE INVENTION

The present invention relates to methods and apparatus for identifying and stabilizing vulnerable plaque. More particularly, the present invention relates to specialized catheters having both an imaging element and a thermographer for improved identification of vulnerable plaque. Apparatus of the present invention may in addition include an optional stabilization element for stabilizing the plaque.

### BACKGROUND OF THE INVENTION

Vulnerable plaque is commonly defined as plaque having a lipid pool with a thin fibrous cap, which is often infiltrated by macrophages. Vulnerable plaque lesions generally manifest only mild to moderate stenoses, as compared to the large stenoses associated with fibrous and calcified lesions. While the more severe stenoses of fibrous and calcified lesions may limit flow and result in ischemia, these larger plaques often remain stable for extended periods of time. In fact, rupture of vulnerable plaque is believed to be responsible for a majority of acute ischemic and occlusive events, including unstable angina, myocardial infarction, and sudden cardiac death.

The mechanism behind such events is believed to be thrombus formation upon rupture and release of the lipid pool contained within vulnerable plaque. Thrombus formation leads to plaque growth and triggers acute events. Plaque rupture may be the result of inflammation, or of lipid accumulation that increases fibrous cap stress. Clearly, prospective identification and stabilization of vulnerable plaque is key to effectively controlling and reducing acute ischemic and occlusive events.

A significant difficulty encountered while attempting to identify and stabilize vulnerable plaque is that standard angiography provides no indication of whether or not a given plaque is susceptible to rupture. Furthermore, since the degree of stenosis associated with vulnerable plaque is often low, in many cases vulnerable plague may not even be visible using angiography.

A variety of techniques for identifying vulnerable plaque are being pursued. These include imaging techniques, for example, Intravascular Ultrasound (“IVUS”), Optical Coherence Tomography (“OCT”), and Magnetic Resonance Imaging (“MRI”). Two primary IVUS techniques have been developed. The first is commonly referred to as rotational IVUS, which uses an ultrasound transducer that is rotated to provide a circumferential image of a patient's vessel. The second technique is commonly referred to as phased-array IVUS, which uses an array of discrete ultrasound elements that each provide image data. The image data from each element is combined to form a circumferential image of the patient's vessel.

Rotational IVUS systems are marketed by the Boston Scientific Corporation of Natick, Mass., and are described, for example, in U.S. Pat. No. 6,221,015 to Yock, which is incorporated herein by reference. Phased-array IVUS systems are marketed by JOMED Inc., of Rancho Cordova, Calif., and are described, for example, in U.S. Pat. No. 6,283,920 to Eberle et al., as well as U.S. Pat. No. 6,283,921 to Nix et al., both of which are incorporated herein by reference. Optical Coherence Tomography systems are developed by Lightlab Imaging, LLC., of Westford, Mass., and are described, for example, in U.S. Pat. No. 6,134,003 to Tearney et al., which is incorporated herein by reference. U.S. Pat. No. 5,699,801 to Atalar et al., which also is incorporated herein by reference, describes methods and apparatus for Magnetic Resonance Imaging inside a patient's vessel.

A primary goal while characterizing plaque-type via an imaging modality is identification of sub-intimal lipid pools at the site of vulnerable plaque. In an IVUS study entitled, “Morphology of Vulnerable Coronary Plaque: Insights from Follow-Up of Patients Examined by Intravascular Ultrasound Before an Acute Coronary Syndrome” (Journal of the American College of Cardiology, 2000; 35:106-11), M. Yamagishi et al., concluded that, “the risk of rupture is high among eccentric lesions with a relatively large plaque burden and a shallow echolucent zone.” IVUS allows characterization of the concentricity or eccentricity of lesions, as well as identification of echolucent zones, which are indicative of lipid-rich cores. However, while IVUS and other advanced imaging modalities may provide a means for identifying vulnerable plaque and selecting patients likely to benefit from aggressive risk factor interventions, such imaging modalities typically require a significant degree of skill, training and intuition on the part of a medical practitioner in order to achieve a proper diagnosis.

In addition to imaging techniques, biological S techniques have also been proposed for identifying vulnerable plaque. Biological techniques typically rely on characterization of material properties of the plaque. Biological techniques include thermography, biological markers, magnetic resonance, elastography and palpography. Biological markers typically attempt to ‘tag’ specific tissue types, for example, via chemical receptors, with markers that allow easy identification of tissue type. Magnetic resonance operates on the principal that different tissue types may resonate at different, identifiable frequencies. Techniques combining Magnetic Resonance Imaging and biological markers have also been proposed in which superparamagnetic iron oxide nanoparticles are used as MRI contrast media. It is expected that vulnerable plaque will preferentially take up the nanoparticles by virtue of macrophage infiltration, leaking vasa vasorum, and permeable thin cap (M. AbouQamar et al., Poster Abstract, Transcatheter Cardiovascular Therapeutics, 2001, Washington, D.C.).

Elastography and palpography seek to characterize the strain modulus, or other mechanical properties, of target tissue. Studies have shown that different plaque types exhibit different, identifiable strain moduli, which may be used to characterize plaque type. Elastography is described, for example, in U.S. Pat. No. 5,178,147 to Ophir et al., which is incorporated herein by reference. Palpography is described, for example, in U.S. Pat. No. 6,165,128 to Cespedes et al., which also is incorporated herein by reference.

Thermography seeks to characterize tissue type via tissue temperature. Tissue temperature may be characterized, for example, via thermographers, thermistors, thermosensors, thermocouples, thermometers, spectrography, spectroscopy, and infrared. Tissue characterization via thermographers has been known for some time; for example, U.S. Pat. No. 4,960,109 to Lele et al., which is incorporated herein by reference, describes a multi-function probe for use in hyperthermia therapy that employs at least one pair of temperature sensors.

It has been observed that vulnerable plaque results in a temperature increase at a vessel wall of as much as about 0.1-1.5° C. A review of thermographic apparatus and techniques for plaque characterization is provided by C. Stefanadis in “Plaque Thermal Heterogeneity—Diagnostic Tools and Management Implications” (Expert Presentation, Transcatheter Cardiovascular Therapeutics, Washington, D.C.). Thermography apparatus and methods are also provided in Greek Patent No. 1003158B to Diamantopoulos et al., Greek Patent No. 1003178B to Toutouzas et al., and Greek Utility Model No. 98200093U to Diamantopoulos et al., all of which are incorporated herein by reference. U.S. Pat. No. 5,445,157 to Adachi et al., which is incorporated herein by reference, describes a thermographic endoscope including an infrared image forming device. U.S. Pat. No. 5,871,449 to Brown and U.S. Pat. No. 5,935,075 to Casscells et al., both incorporated herein by reference, describe catheters capable of detecting infrared radiation.

Although passing reference is made in the Abstract of the Casscells patent to using the infrared detection system with or without ultrasound, no ultrasound apparatus is described. If ultrasound were to be used, it would presumably be applied using known techniques, i.e. extravascularly or via a secondary, stand-alone IVUS catheter. Using extravascular ultrasound or a secondary, stand-alone IVUS catheter, in conjunction with an infrared catheter is expected to increase the complexity, time, and cost associated with identifying vulnerable plaque.

For the purposes of the present invention, in addition to temperature characterization, thermography includes characterization of tissue pH, for example, via Near-Infrared (“NIR”) Spectroscopy. T. Khan et al., have shown that inflamed regions of plaque exhibit lower pH, and that NIR Spectroscopy may be used to measure such pH (“Progress with the Calibration of A 3-French Near Infrared Spectroscopy Fiberoptic Catheter for Monitoring the pH Of Atherosclerotic Plaque: Introducing a Novel Approach For Detection of Vulnerable Plaque,” Poster Abstract, Transcatheter Cardiovascular Therapeutics, 2001, Washington, D.C.).

Although thermography is a promising new technique for identifying vulnerable plaque, it has several drawbacks. First, since thermography doesn't provide image data, it is expected that medical practitioners will have difficulty determining proper locations at which to use a thermographer in order to characterize plaque type. Thus, secondary, stand-alone imaging apparatus may be required in order to adequately identify and characterize plaque. Requiring separate imaging and thermography apparatus is expected to increase complexity, time and cost associated with identifying vulnerable plaque. Additionally, thermography provides no indication of the eccentricity of a plaque or of the presence or magnitude of lipid pools disposed in the plaque, both of which have been shown to indicate the presence of vulnerable plaque.

A drawback common to prior art techniques for identifying and stabilizing vulnerable plaque is that identification and stabilization are typically achieved using separate apparatus. Stabilization techniques include both local and systemic therapy. Localized techniques include angioplasty, stenting, mild heating, photonic ablation, radiation, local drug injection, gene therapy, covered stents and coated stents, for example, drug-eluting stents. Systemic therapies include extreme lipid lowering; inhibition of cholesterol acyltransferase (Acyl-CoA, “ACAT”); matrix metalloproteinase (“MMP”) inhibition; and administration of anti-inflammatory agents, anti-oxidants and/or Angiotensin-Converting Enzyme (“ACE”) inhibitors.

Multi-functional devices have been proposed in other areas of vascular intervention. For example, U.S. Pat. No. 5,906,580 to Kline-Schoder et al., which is incorporated herein by reference, describes an ultrasound transducer array that may transmit signals at multiple frequencies and may be used for both ultrasound imaging and ultrasound therapy. PharmaSonics, Inc., of Sunnyvale, Calif., markets therapeutic ultrasound catheters, which are described, for example, in U.S. Pat. No. 5,725,494 to Brisken et al., incorporated herein by reference. U.S. Pat. No. 5,581,144 to Corl et al., incorporated herein by reference, describes another ultrasound transducer array that is capable of operating at multiple frequencies.

In addition to multi-functional ultrasound devices, other multi-functional interventional devices are described in U.S. Pat. Nos. 5,571,086 and 5,855,563 to Kaplan et al., both of which are assigned to Localmed, Inc., of Palo Alto, Calif., and both of which are incorporated herein by reference. However, none of these devices, nor the multi-functional ultrasound devices discussed previously, are suited for rapid identification and stabilization of vulnerable plaque in accordance with the principles of the present invention.

In view of the drawbacks associated with previously known methods and apparatus for identifying and stabilizing vulnerable plaque, it would be desirable to provide methods and apparatus that overcome those drawbacks.

It would be desirable to provide methods and apparatus that reduce the skill and training required on the part of medical practitioners in order to identify and stabilize vulnerable plaque.

It would be desirable to provide methods and apparatus for identifying and stabilizing vulnerable plaque that reduce the cost, complexity and time associated with such procedures.

It would be desirable to provide methods and apparatus that are multi-functional.

It would be desirable to provide methods and apparatus that facilitate characterization of lesion eccentricity, echogenicity, and temperature or pH.

It would be desirable to provide methods and apparatus that combine imaging, thermography and, optionally, vulnerable plaque stabilization elements in a single device.

### SUMMARY OF THE INVENTION

In view of the foregoing, it is an object of the present invention to provide apparatus and methods for identifying and stabilizing vulnerable plaque that overcome drawbacks associated with previously known apparatus and methods.

It is an object to provide methods and apparatus that reduce the skill and training required on the part of medical practitioners in order to identify and stabilize vulnerable plaque.

It also is an object to provide methods and apparatus for identifying and stabilizing vulnerable plaque that reduce the cost, complexity and time associated with such procedures.

It is another object to provide methods and apparatus that are multi-functional.

It is yet another object to provide methods and apparatus that facilitate characterization of lesion eccentricity, echogenicity, and temperature or pH.

It is an object to provide methods and apparatus that combine imaging, thermography and, optionally, vulnerable plaque stabilization elements in a single device.

These and other objects of the present invention are accomplished by providing apparatus for identifying vulnerable plaque comprising a catheter having both an imaging element and a thermographer. Providing both thermography and imaging in a single, multi-functional catheter is expected to decrease the cost and increase the accuracy of vulnerable plaque identification, as well as simplify and expedite identification, as compared to providing separate, standalone thermography and imaging. Apparatus of the present invention also may be provided with optional stabilization elements for stabilizing vulnerable plaque, thereby providing vulnerable plaque identification and stablization in a single device.

In a first embodiment of the present invention, a catheter is provided having a phased-array IVUS imaging system and a plurality of thermocouples. The plurality of thermocouples may be deployed into contact with an interior wall of a patient's body lumen, thereby providing temperature measurements along the interior wall that may be compared to IVUS images obtained with the imaging system to facilitate identification of vulnerable plaque. In a second embodiment, a catheter is provided with a rotational IVUS imaging system and a fiber optic infrared thermography system. The infrared system's fiber optic is preferably coupled to the rotating drive cable of the rotational IVUS imaging system, thereby providing a full circumferential temperature profile along the interior wall of the patient's body lumen.

In a third embodiment, apparatus of the present invention is provided with, in addition to an imaging element and a thermographer, an optional stabilization element. The stabilization element comprises an inflatable balloon. In a fourth embodiment, the stabilization element comprises a second ultrasound transducer that resonates at therapeutic ultrasound frequencies, as opposed to ultrasonic imaging frequencies. As yet another embodiment, the imaging element of the present invention comprises an ultrasound transducer that is capable of transmitting multiple frequencies that are suited to both ultrasonic imaging and ultrasonic therapy, thereby providing both vulnerable plaque imaging and stabilization in a single element. These embodiments are provided only for the purpose of illustration. Additional embodiments will be apparent to those skilled in the art and are included in the scope of the present invention.

Imaging and thermographic data are preferably coupled in order to facilitate identification of vulnerable plaque. Coupling may be achieved using position indication techniques, for example, using an IVUS pullback system that is modified to simultaneously monitor the position of both the imaging element and the thermographer. IVUS pullback systems are described, for example, in U.S. Pat. No. 6,290,675 to Vujanic et al., U.S. Pat. No. 6,275,724 to Dickinson et al., U.S. Pat. No. 6,193,736 to Webler et al., and PCT Publication WO 99/12474, all of which are incorporated herein by reference.

Imaging data and thermographic data, coupled using position indication techniques, are preferably simultaneously graphically displayed, for example, on a standard computer monitor. The coupled data is preferably displayed in an overlayed fashion so that a medical practitioner may rapidly correlate temperature measurements obtained at a given position within the patient's body lumen to images obtained at that position. Rapid correlation is expected to simplify, expedite and increase the accuracy of vulnerable plaque identification, as well as facilitate plaque stabilization. It is expected that additional data also may be obtained, coupled and provided in the graphical display, for example, palpography data. Blood flow imaging, as described, for example, in U.S. Pat. Nos. 5,453,575 and 5,921,931 to O'Donnell et al., both of which are incorporated herein by reference, may also be provided.

Methods of using the apparatus of the present invention are also provided.

### BRIEF DESCRIPTION OF THE DRAWINGS

Further features of the invention, its nature and various advantages, will be more apparent from the following detailed description of the preferred embodiments, taken in conjunction with the accompanying drawings, in which like reference numerals apply to like parts throughout, and in which:

FIG. 1 is a schematic cut-away view of a prior art phased-array IVUS catheter;

FIG. 2 is a schematic cut-away view of a prior art rotational IVUS catheter;

FIGS. 3A and 3B are schematic side views of a prior art thermography catheter having a plurality of thermocouples, and shown in a collapsed delivery configuration and an expanded deployed configuration, respectively;

FIG. 4 is a schematic cut-away view of a prior art thermography catheter having a side-viewing infrared thermographer;

FIG. 5 is a schematic side view of a prior art thermography catheter having a steerable distal region with a thermocouple;

FIG. 6 is a schematic side view of a first embodiment of a catheter in accordance with the principles of the present invention having an imaging element and a thermographer;

FIG. 7 is a schematic cut-away view of a second embodiment of apparatus of the present invention having an imaging element and a thermographer;

FIG. 8 is a schematic side view of a third embodiment of apparatus in accordance with the present invention having an optional stabilization element;

FIG. 9 is a schematic side view of a fourth embodiment of the present invention having an alternative stabilization element;

FIGS. 10A and 10B are schematic side views, partially in section, of the apparatus of FIG. 7 disposed at a target site within a patient's vessel, illustrating a method of using the apparatus of the present invention;

FIGS. 11A and 11B are schematic views of graphical user interfaces that display imaging and thermographic data, respectively, obtained, for example, via the method of FIGS. 10, with the thermographic data of FIG. 11B obtained along side-sectional view line A--A of FIG. 11A;

FIG. 12 is a schematic view of a graphical user interface that couples and simultaneously displays imaging and thermographic data obtained along a cross-section of the patient's vessel; and

FIG. 13 is a schematic view of an alternative graphical user interface that simultaneously displays coupled imaging and thermographic data along side-sectional view line B--B of FIG. 12.

### DETAILED DESCRIPTION OF THE INVENTION

The present invention relates to methods and apparatus for identifying and stabilizing vulnerable plaque. More particularly, the present invention relates to specialized catheters having both an imaging element and a thermographer for improved identification of vulnerable plaque. Apparatus of the present invention may in addition include an optional stabilization element for stabilizing the plaque.

With reference to FIG. 1, a prior art phased-array Intravascular Ultrasound (“IVUS”) catheter is described. Catheter 10 comprises phased-array ultrasound transducer 12 having a plurality of discrete ultrasound elements 13. Catheter 10 further comprises guide wire lumen 14, illustratively shown with guide wire 100 disposed therein. Catheter 10 also may comprise multiplexing circuitry, amplifiers, etc., per se known, which may be disposed on and/or electrically coupled to catheter 10. Transducer array 12 of catheter 10 is electrically coupled to an imaging system (not shown), per se known, that provides excitation waveforms to the transducer array, and interprets and displays data received from the array.

FIG. 2 depicts a prior art rotational IVUS catheter. Catheter 20 comprises ultrasound transducer 22 disposed on a distal region of rotatable drive cable 24. Drive cable 24 is proximally coupled to a driver (not shown), e.g. an electric motor, for rotating the drive cable and ultrasound transducer 22, thereby providing transducer 22 with a 360° view. Catheter 20 further comprises guide wire lumen 26 that opens in side port 28 distally of transducer 22. Guide wire 100 is illustratively disposed within lumen 26. As with transducer array 12 of catheter 10, transducer 22 of catheter 20 is electrically coupled to an imaging system (not shown), per se known, that provides excitation waveforms to the transducer, and interprets and displays data received from the transducer.

As discussed hereinabove, it has been shown that sub-intimal lipid pools at the site of plaque, as well as the eccentricity of the plaque, are key indicators of vulnerable plaque susceptible to rupture. It has also been shown that IVUS may be used to determine the eccentricity of plaque, as well as to identify echolucent zones, which are indicative of lipid-rich cores. However, achieving proper identification of vulnerable plaque via IVUS or any of a host of other advanced imaging modalities (e.g. Magnetic Resonance Imaging or Optical Coherence Tomography) may require a significant degree of skill, training and intuition on the part of a medical practitioner.

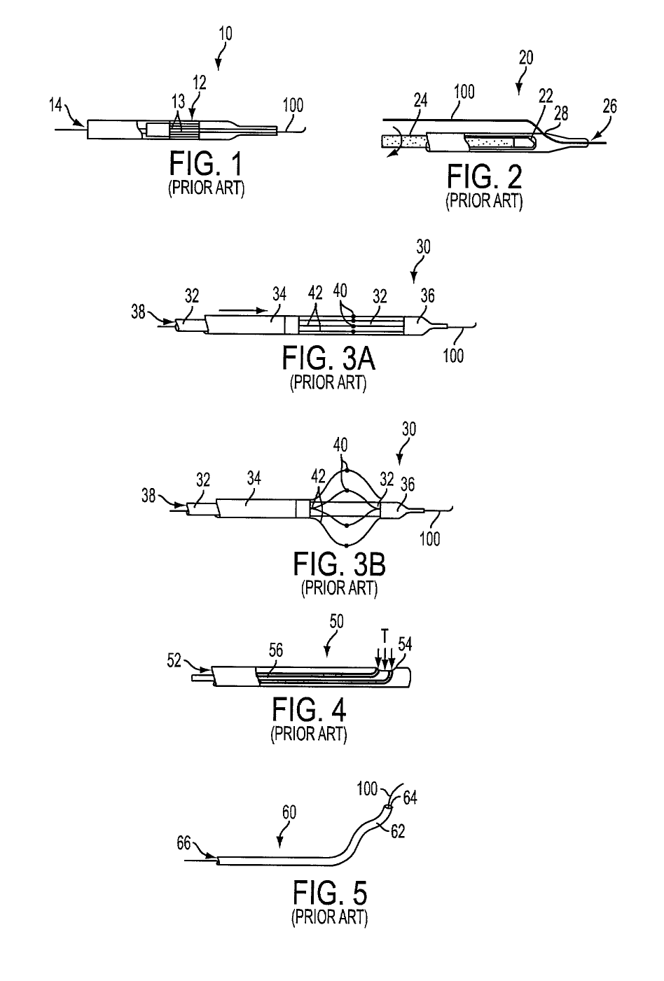

With reference now to FIGS. 3, a prior art thermography catheter is described. Catheter 30 comprises outer tube 34 coaxially disposed about inner tube 32. Inner tube 32 comprises distal tip 36 and guide wire lumen 38, in which guide wire 100 is illustratively disposed. Catheter 30 further comprises a plurality of thermocouples 40 disposed near its distal end. Each thermocouple comprises a wire 42 coupled proximally to the distal end of outer tube 34 and distally to distal tip 36 of inner tube 32. The proximal and distal ends of each wire 42 are further electrically coupled to a processor (not shown) that captures and translates voltages generated by thermocouples 40 into temperature values, for example, via known calibration values for each thermocouple.

As seen in FIG. 3, catheter 30 is expandable from the collapsed delivery configuration of FIG. 3A to the expanded deployed configuration of FIG. 3B, by advancing outer tube 34 with respect to inner tube 32. Such advancement causes thermocouples 40 to protrude from catheter 30 so that the thermocouples may contact the interior wall of a patient's body lumen. Catheter 30 is adapted for intravascular delivery in the collapsed configuration of FIG. 3A, and is adapted for taking temperature measurements at a vessel wall in the expanded configuration of FIG. 3B.

Referring to FIG. 4, another prior art thermography catheter is described. Catheter 50 comprises lumen 52, which extends from a proximal end of catheter 50 to distal side port 54. Fiber optic 56 is disposed within lumen 52 and is proximally coupled to an infrared thermography system (not shown). Catheter 50 thereby comprises a side-viewing fiber optic thermography catheter capable of measuring ambient temperature T near distal side port 54.

By disposing side port 54 of catheter 50 within a patient's body lumen, the temperature of the patient's body lumen may be measured to facilitate identification of vulnerable plaque. However, a significant drawback of catheter 50 for identification of vulnerable plaque is that fiber optic 56 has only a limited field of view, and vulnerable plaque is typically eccentric, i.e. occurs predominantly on one side of a vessel. Thus, if side port 54 of catheter 50 were not rotated to the side of the vessel afflicted with vulnerable plaque build-up, it is expected that the ambient temperature T measured with catheter 50 would not reflect the presence of vulnerable plaque.

With reference to FIG. 5, yet another prior art thermography catheter is described. Catheter 60 comprises steerable distal end 62 having thermistor 64 coupled thereto. Thermistor 64 is proximally attached to a processor (not shown) that converts measurements taken with thermistor 64 into temperature measurements. Catheter 60 further comprises guide wire lumen 66 having guide wire 100 illustratively disposed therein.

Distal end 62 of catheter 60 may be positioned against a patient's body lumen to provide temperature measurements where thermistor 64 contacts the body lumen. However, a significant drawback of catheter 60 is that thermistor 64 only provides temperature measurements at a single point at any given time. It is therefore expected that eccentric vulnerable plaque will be difficult to identify with catheter 60, especially when distal end 62 of catheter 60 is disposed against the unaffected, or mildly affected, side of a patient's vessel suffering from eccentric vulnerable plaque.

As discussed previously, although thermography is a promising new technique for identifying vulnerable plaque, all the thermography devices described hereinabove have several drawbacks. First, since thermography doesn't provide image data, it is expected that medical practitioners will have difficulty determining proper locations at which to use a thermographer in order to characterize plaque type. Thus, secondary, stand-alone imaging apparatus may be required in order to adequately identify and characterize plaque. Requiring separate imaging and thermography apparatus is expected to increase complexity, time and cost associated with identifying vulnerable plaque. Additionally, thermography provides no indication of the eccentricity of a plaque or of the presence or magnitude of lipid pools disposed in the plaque, both of which have been shown to indicate the presence of vulnerable plaque.

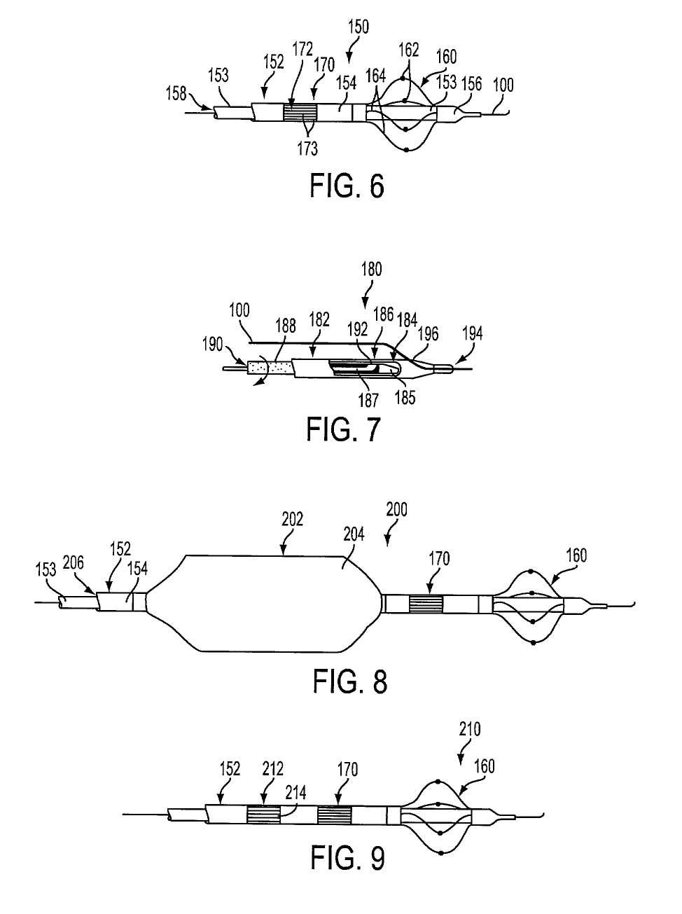

With reference now to FIG. 6, a first embodiment of apparatus in accordance with the present invention is described that provides both an imaging element and a thermographer in a single device. By providing both imaging and thermography in a single device, the present invention combines positive attributes of stand-alone imaging systems and stand-alone thermographers described hereinabove, while reducing previously-described drawbacks associated with such stand-alone systems. Apparatus 150 of FIG. 6 comprises catheter body 152, thermographer 160 and imaging element 170.

Catheter body 152 comprises outer tube 154 coaxially disposed about inner tube 153. Inner tube 153 comprises distal tip 156 and guide wire lumen 158, in which guide wire 100 is illustratively disposed. Thermographer 160 comprises a plurality of thermocouples 162. Any number of thermocouples 162 may be provided. Each thermocouple comprises a wire 164 coupled proximally to the distal end of outer tube 154 and distally to distal tip 156 of inner tube 153. The proximal and distal ends of each wire 164 are further electrically coupled to a processor (not shown) that captures and translates voltages generated by thermocouples 162 into temperature values, for example, via known calibration values for each thermocouple.

Imaging element 170 comprises phased-array ultrasound transducer 172 having a plurality of discrete ultrasound elements 173. Imaging element 170 optionally may comprise multiplexing circuitry, flexible circuitry or substrates, amplifiers, etc., per se known, which may be disposed on and/or electrically coupled to apparatus 150. Transducer array 172 of imaging element 170 is electrically coupled to an imaging system (not shown), per se known, that provides excitation waveforms to the transducer array, and interprets and displays data received from the array. The imaging system coupled to imaging element 170 and the processor coupled to thermographer 160 are preferably combined into a single data acquisition and analysis system (not shown) for capturing and interpreting data received from apparatus 150.

As with catheter 30 of FIGS. 3, apparatus 150 is expandable from a collapsed delivery configuration to the expanded deployed configuration of FIG. 6, by advancing outer tube 154 of catheter body 152 with respect to inner tube 153. Such advancement causes thermocouples 162 of thermographer 160 to protrude from catheter body 152 so that the thermocouples may contact the interior wall of a patient's body lumen. Apparatus 150 is adapted for intravascular delivery in the collapsed configuration, and is adapted for taking temperature measurements at a vessel wall in the expanded configuration. Imaging via imaging element 170 may be achieved in either the collapsed delivery configuration or the expanded deployed configuration, thereby facilitating positioning of apparatus 150 at a stenosed region within a patient's vessel.

Thermographer 160 comprises multiple thermography sensors, illustratively in the form of thermocouples 162, disposed radially about catheter body 152. Temperature measurements obtained from these sensors may be displayed graphically as a 2-dimensional map or image, for example, as a cross-sectional temperature profile within a patient's vessel. Such a cross-sectional temperature profile may be compared with a cross-sectional image of the vessel obtained at the same location, for example, via imaging element 170. By advancing or retracting catheter body 152, this 2-dimensional map, as well as the cross-sectional image, may be extended to 3-dimensions. Translation of catheter body 152 may be achieved, for example, using position indication techniques and/or a pullback system, per se known. Illustrative methods and apparatus for displaying thermographic and imaging data are provided hereinbelow with respect to FIGS. 11-13.

Apparatus 150 is expected to provide significant advantages over prior art, stand-alone imaging and thermography catheters, such as catheters 10 and 30, used either alone or in combination. Specifically, apparatus 150 is expected to decrease the complexity of obtaining both temperature and imaging data at a target site, as well as to facilitate correlation of such data. Additionally, apparatus 150 is expected to reduce the cost of obtaining both temperature and imaging data, as compared to providing both a stand-alone imaging system and a stand-alone thermography system.

Since vascular lumens commonly afflicted with vulnerable plaque, such as the coronary arteries, are often very small, it is expected that difficulty may be encountered while trying to simultaneously position separate imaging and thermography catheters at the site of vulnerable plaque; furthermore, a stand-alone thermography catheter may block imaging of portions of the vessel wall. Apparatus 150 overcomes these drawbacks. Additionally, apparatus 150 is expected to reduce the skill required on the part of a medical practitioner to identify vulnerable plaque via IVUS, by providing a secondary indication of vulnerable plaque in the form of temperature measurements. Likewise, apparatus 150 is expected to increase the likelihood of proper vulnerable plaque identification via thermography, by providing a secondary indication of vulnerable plaque in the form of IVUS imaging that allows examination of plaque eccentricity and echogenicity. Additional advantages of the present invention will be apparent to those of skill in the art.

Referring now to FIG. 7, a second embodiment of apparatus in accordance with the present invention in described. Apparatus 180 comprises catheter 182 having imaging element 184 and thermographer 186. Imaging element 184 comprises a rotational IVUS imaging element, and thermographer 186 comprises a rotational infrared thermographer.

Catheter 182 further comprises rotatable drive cable 188 having lumen 190 that distally terminates at side port 192. Catheter 182 still further comprises guide wire lumen 194 that opens in side port 196 distally of drive cable 188. Guide wire 100 is illustratively shown disposed in lumen 194.

Thermographer 186 of catheter 182 comprises fiber optic 187 disposed within lumen 190 of drive cable 188. Imaging element 184 of catheter 182 comprises ultrasound transducer 185 disposed on rotatable drive cable 188. Drive cable 188 is proximally coupled to a driver (not shown), e.g. an electric motor, for rotating the drive cable, as well as ultrasound transducer 185 of imaging element 184 and fiber optic 187 of thermographer 186, thereby providing imaging element 184 and thermographer 186 with a 360° view.

As with transducer 22 of catheter 20, transducer 185 is electrically coupled to an imaging system (not shown), per se known, that provides excitation waveforms to the transducer, and interprets and displays data received from the transducer. Likewise, as with fiber optic 56 of catheter 50, fiber optic 187 is proximally coupled to an infrared thermography system (not shown). Preferably, the imaging system of imaging element 184, the infrared thermography system of thermographer 186, and the driver coupled to drive cable 188, are combined into a single data acquisition and analysis system (not shown) for capturing and interpreting data received from apparatus 180. Alternatively, a subset of these elements may be combined.

Apparatus 180 provides many of the advantages described hereinabove with respect to apparatus 150. Additionally, as compared to infrared thermography catheter 50, described hereinabove with respect to FIG. 4, thermographer 186 of apparatus 180 provides significantly enhanced thermographic capabilities. Specifically, by coupling thermographer 186 to rotatable drive cable 188, thermographer 186 is capable of providing a full circumferential temperature profile along the interior wall of a patient's body lumen, without necessitating potentially inaccurate manual rotation of the infrared thermographer by a medical practitioner. A stand-alone, rotatable infrared thermography catheter (not shown), similar to apparatus 180 but without imaging capabilities, is contemplated and is included in the scope of the present invention.

With reference to FIG. 8, a third embodiment of apparatus in accordance with the present invention is described that includes an optional stabilization element, in addition to an imaging element and a thermographer. The stabilization element is adapted to stabilize vulnerable plaque, thereby providing vulnerable plaque identification and stablization in a single device. Apparatus 200 comprises all of the elements of apparatus 150, including catheter body 152, thermographer 160 and imaging element 170, and further comprises stabilization element 202.

Stabilization element 202 comprises inflatable balloon 204. Balloon 204 is inflatable from a collapsed delivery configuration to the deployed configuration of FIG. 8 by suitable means, for example, via an inflation medium injected into the balloon through annulus 206 formed between the inner wall of outer tube 154 and the outer wall of inner tube 153 of catheter body 152. Additional inflation techniques will be apparent to those skilled in the art.

It is expected that, once vulnerable plaque has been identified in a patient's vessel via thermographer 160 and/or imaging element 170, stabilization element 202 may be positioned at the location of the identified vulnerable plaque. Stabilization element 202 may then be deployed, i.e. balloon 204 may be inflated, at the site of vulnerable plaque to stabilize the plaque, for example, by compressing, rupturing, scaffolding and/or sealing the plaque in the controlled environment of a catheterization laboratory. In addition to balloon 204, stabilization element 202 may be provided with additional stabilization elements (not shown), for example, a stent, a covered stent, a stent graft, a coated stent, or a drug-eluting stent, to further enhance stabilization of vulnerable plaque. Additional stabilization elements will be apparent to those of skill in the art.

In order to facilitate identification and stabilization of vulnerable plaque, the distances between stabilization element 202, thermographer 160 and imaging element 170 are preferably provided or measured. Furthermore, the distances between the imaging, thermography and optional stabilization elements of all embodiments of the present invention are preferably provided or measured. This facilitates coupling of thermographic and imaging data, as well as proper positioning of optional stabilization elements.

Providing vulnerable plaque identification and stabilization elements in a single device, in accordance with the principles of the present invention, provides all of the benefits of apparatus 150 described hereinabove, as well as the additional advantage of not having to provide stand-alone apparatus for plaque stabilization. This, in turn, is expected to decrease the cost, time and complexity associated with identifying and stabilizing vulnerable plaque, as well as to decrease the crossing profile of such apparatus, as compared to stand-alone apparatus used concurrently. Further still, providing identification and stabilization in a single device is expected to simplify accurate placement of stabilization elements at the site of identified vulnerable plaque.

Referring now to FIG. 9, a fourth embodiment of the present invention having an alternative vulnerable plaque stabilization element, is described. Apparatus 210 comprises all of the elements of apparatus 150, including catheter body 152, thermographer 160 and imaging element 170, and further comprises stabilization element 212. Stabilization element 212 comprises therapeutic ultrasound transducer 214, which is capable of resonating at, and transmitting, therapeutic ultrasound frequencies. Transducer 214 may comprise a single element or an array of elements. Transducer 214 is attached to an excitation unit (not shown) capable of causing resonance within the transducer. The excitation unit is preferably combined with the imaging system (not shown) of imaging element 170.

Therapeutic ultrasound frequencies, at which therapeutic transducer 214 preferably is capable of resonating and transmitting, are typically described as low frequencies, for example, frequencies below 10,000,000 Hertz, or 10 Megahertz (“MHz”), and even more preferably frequencies below about 500,000 Hertz, or 500 Kilohertz (“kHz”). Conversely, transducer array 172 of imaging element 170 preferably is capable of resonating at, and transmitting, imaging ultrasound frequencies. Imaging ultrasound frequencies are typically described as high frequencies, for example, frequencies above about 10 Megahertz (“MHz”). These frequencies are provided only for the sake of illustration and should in no way be construed as limiting.

It is expected that, once vulnerable plaque has been identified in a patient's vessel via thermographer 160 and/or imaging element 170, stabilization element 212 may be positioned at the location of the identified plaque and activated, i.e. ultrasound transducer 214 may provide therapeutic ultrasound waves, to stabilize the plaque, for example, by compressing, rupturing, and/or sealing the plaque in the controlled environment of a catheterization laboratory. As with apparatus 200, the distances between stabilization element 212, thermographer 160 and imaging element 170 are preferably provided or measured in order to facilitate vulnerable plaque identification, as well as positioning of stabilization element 212 prior to activation.

In addition to therapeutic ultrasound transducer 214, stabilization element 212 may be provided with additional stabilization elements (not shown), for example, contrast, tissue-tag, or therapeutic agents, such as drug capsules, that rupture and are released upon exposure to ultrasound waves generated by therapeutic ultrasound transducer 214. Additional stabilization elements will be apparent to those of skill in the art. Apparatus 210 is expected to provide many of the benefits described hereinabove with respect to apparatus 150 and apparatus 200.

As yet another embodiment of the present invention, apparatus may be provided in which imaging element 170 and stabilization element 212 of apparatus 210 are replaced with a single ultrasonic transducer array that is capable of transmitting multiple frequencies suited to both ultrasonic imaging and ultrasonic therapy, thereby providing both vulnerable plaque imaging and stabilization in a single element. Techniques for providing an ultrasound transducer capable of resonating at multiple frequencies are provided, for example, in U.S. Pat. No. 5,906,580 to Kline-Schoder et al., as well as U.S. Pat. No. 5,581,144 to Corl et al., both of which are incorporated herein by reference.

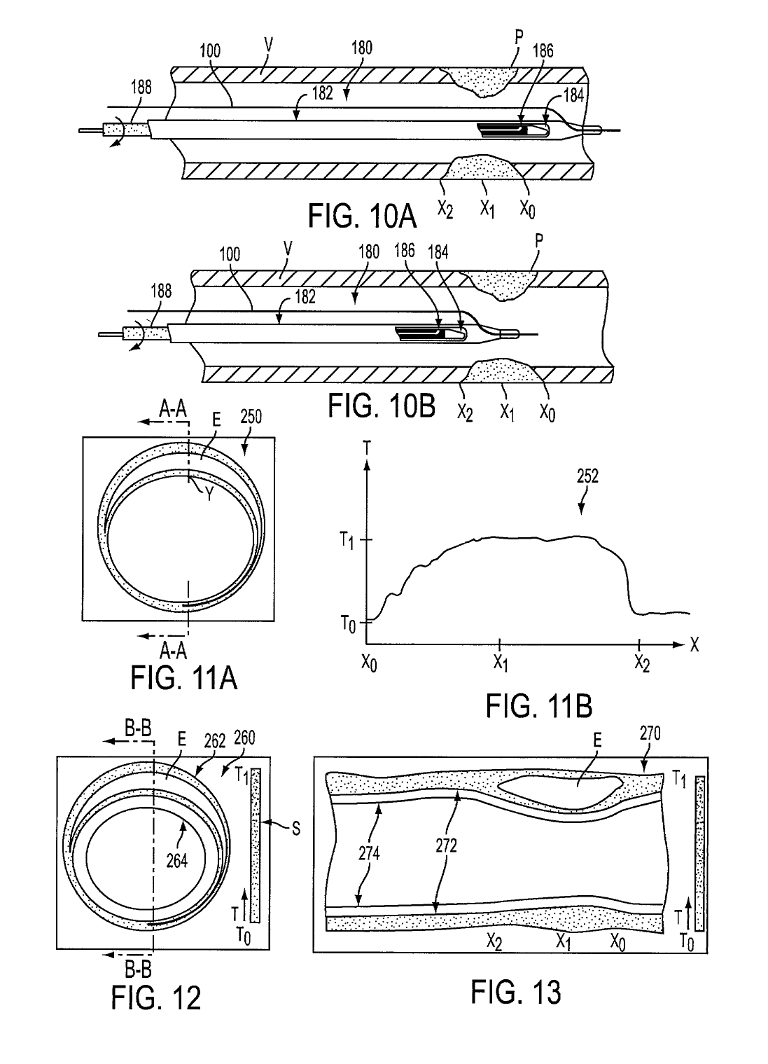

With reference to FIG. 10, a method of using apparatus of the present invention is provided, illustratively using apparatus 180 described hereinabove. In FIG. 10, vessel V is afflicted with eccentric vulnerable plaque P that manifests only mild stenosis within vessel V. Catheter 182 of apparatus 180 is percutaneously advanced into vessel V, for example, over guide wire 100, such that imaging element 184 and thermographer 186 are disposed distally of distal edge x₀ of vulnerable plaque P, as seen in FIG. 10A. Drive cable 188 is rotated via its driver (not shown) such that imaging element 184 and thermographer 186 are provided with a full 360° view.

Catheter 182 is then withdrawn proximally across the stenosis until imaging element 184 and thermographer 186 are disposed proximally of proximal edge x₂ of vulnerable plaque P, as seen in FIG. 10B. Imaging and thermography data are collected via imaging element 184 and thermographer 186, respectively, during proximal retraction of catheter body 182 across the stenosis. Proximal retraction may be achieved manually or using a pullback system. Pullback systems are described, for example, in U.S. Pat. No. 6,290,675 to Vujanic et al., U.S. Pat. No. 6,275,724 to Dickinson et al., U.S. Pat. No. 6,193,736 to Webler et al., and PCT Publication WO 99/12474, all of which are incorporated herein by reference.

As will be apparent to those of skill in the art, catheter 182 alternatively may be advanced distally across vulnerable plaque P during data acquisition, or catheter 182 may be held stationary at a location of interest, for example, location x₁ in the middle of vulnerable plaque P. Additionally, when vulnerable plaque P has been identified, apparatus 180 optionally may be provided with stabilization elements capable of compressing, rupturing, sealing, scaffolding and/or otherwise treating the plaque in the controlled environment of a catheterization laboratory. Exemplary stabilization elements include balloon 204 of apparatus 200, and therapeutic ultrasound transducer 214 of apparatus 210. Additional stabilization elements will be apparent to those of skill in the art.

With reference now to FIG. 11, in conjunction with FIG. 10, graphical user interfaces for displaying and interpreting imaging and thermography data, collected, for example, using the methods of FIG. 10, are described. FIG. 11A provides cross-sectional IVUS image 250 formed from imaging data obtained at location x₁ within the patient's vessel V. Image 250 is eccentric and comprises echolucent zone E, which is indicative of a shallow lipid pool. Both the eccentricity and echogenicity of image 250 are indicative of vulnerable plaque P, with increased risk of rupture, at location x₁ within vessel V.

FIG. 11B displays temperature measurements T as a function of position x. Graphing temperature as a function of position requires that the position of the thermographer be recorded. Such position indication may be achieved, for example, using a pullback system, such as those described hereinabove.

In FIG. 11B, temperature measurements are obtained and graphed along point Y of section line A--A in FIG. 11A during proximal retraction of catheter 182 within vessel V from distal edge x₀ to location x₁ to proximal edge x₂ of vulnerable plaque P. The reference temperature within vessel V at locations proximal and distal of vulnerable plaque P is approximately T₀. All temperatures may be provided as a relative change in temperature with respect to reference temperature T₀, or temperatures may be provided on an absolute scale, as in FIG. 11B.

As seen in graph 252, as catheter 182 is proximally retracted across vulnerable plaque P, the temperature at the interior wall of vessel V along point Y rises from reference temperature T₀ to local maximum temperature T₁. Temperature T₁ is obtained at location x₁ within vessel V. The temperature within the vessel recedes back to reference temperature T₀ while catheter body 182 is further retracted from location x₁ to proximal edge x₂ of vulnerable plaque P. The increase in temperature from reference temperature T₀ to temperature T₁ in the region surrounding location x₁ within the vessel may be as much as 0.1-1.5° C. This range is provided only for the purpose of illustration and should in no way be construed as limiting.

The increase in temperature from T₀ to T₁ is indicative of vulnerable plaque susceptible to rupture. By comparing and correlating the thermographic data of graph 252 of FIG. 11B to IVUS image 250 of FIG. 11A, identification of vulnerable plaque P is corroborated and confirmed. Thus, providing both imaging and thermography simplifies vulnerable plaque identification while reducing a level of skill required on the part of a medical practitioner in order to properly diagnose such plaque.

In addition to graphing temperature measurements as a function of position, temperature measurements may alternatively be displayed as dynamic, individual measurements (not shown) obtained at the current position of the thermographer. As yet another alternative, temperature measurements may be displayed for an entire vessel cross-section (see FIG. 12), such as a cross-section of temperature measurements obtained at location x₁. Cross-sections of thermography and imaging data at a given position may be compared to provide rapid and proper identification of vulnerable plaque.

Referring now to FIG. 12, a graphical user interface for concurrently displaying both imaging and thermography data is described. In FIG. 12, imaging and thermography data are correlated and coupled prior to display, for example, using position indication techniques and/or a pullback system, such as an IVUS pullback system that is modified to simultaneously monitor the position of both the imaging element and the thermographer. Optional stablization elements may also be monitored via position indication techniques and/or a pullback system. IVUS pullback systems are described hereinabove.

In FIG. 12, imaging and thermography data, coupled using position indication techniques, are simultaneously displayed in a graphical, overlayed fashion, for example, on a standard computer monitor. Graphical user interface 260 comprises imaging cross-section 262 and thermography cross-section 264. Both imaging cross-section 262 and thermography cross-section 264 were obtained at location x₁ within vessel V. Imaging cross-section 262 is eccentric and contains echolucent zone E, which is indicative of a shallow lipid pool.

Thermography cross-section 264 is displayed with reference to temperature intensity scale S that ranges between T₀ and T₁. Scale S may be provided as a color shift, an intensity shift, or a combination thereof. Furthermore the line width along thermography cross-section 264 may be altered to indicate changes in temperature. Additionally, the range of scale S may be extended beyond T₀ and T₁, or may be displayed as a change in temperature ΔT from a reference background temperature, such as T₀. Additional scales S will be apparent to those of skill in the art and are included in the present invention. As can be seen in FIG. 12, the intensity of thermography cross-section 264, and thus the temperature within vessel V, increases along eccentric echolucent zone E of imaging cross-section 262, which is indicative of vulnerable plaque.

Overlaying imaging and thermography data facilitates rapid correlation of the temperature at a given position within vessel V to the image obtained at that position. Rapid correlation is expected to simplify, expedite and increase the accuracy of vulnerable plaque identification. Additional data may also be obtained, coupled and provided in the graphical display, for example, palpography data (not shown). Palpographic techniques are described, for example, in U.S. Pat. No. 6,165,128 to Cespedes et al., which is incorporated herein by reference. Blood flow imaging may also be provided (not shown). Blood flow imaging is described, for example, in U.S. Pat. Nos. 5,453,575 and 5,921,931 to O'Donnell et al., both of which are incorporated herein by reference.

Referring now to FIG. 13, an alternative graphical user interface that simultaneously displays coupled imaging and thermography data is described. Graphical user interface 270 overlays imaging and thermography data in a manner similar to interface 260 of FIG. 12. However, interface 270 displays data obtained along side-sectional view line B--B of FIG. 12 during retraction or advancement of apparatus of the present invention across vulnerable plaque P. Retraction or advancement across plaque P is preferably achieved using a modified IVUS pullback system, as described hereinabove.

Graphical user interface 270 comprises imaging side-section 272 and thermography side-section 274. Imaging side-section 272 is eccentric and comprises echolucent zone E, which is most pronounced in the region around location x₁ within vessel V. Likewise, thermography side-section 274 is of greatest intensity in the region around echolucent zone E of imaging side-section 272. Concurrent analysis of imaging side-section 272 and correlated thermography side-section 274 is expected to facilitate improved identification of vulnerable plaque. As with the cross-sectional view of graphical user interface 260 of FIG. 12, additional information, for example, palpography information or blood flow information, may be provided within the side-sectional view of graphical user interface 270, in order to further facilitate plaque identification. The additional data, e.g. the palpography data or the blood flow data, is preferably obtained concurrently with imaging data, for example, via the imaging element.

While preferred illustrative embodiments of the present invention are described hereinabove, it will be apparent to those of skill in the art that various changes and modifications may be made therein without departing from the invention. For example, the specific structure of the imaging elements, thermographers, and stabilization elements of the embodiments of FIGS. 6-10, are provided only for the sake of illustration. Contemplated imaging elements include, but are not limited to, ultrasound transducers, linear-array ultrasound transducers, phased-array ultrasound transducers, rotational ultrasound transducers, forward-looking ultrasound transducers, radially-looking ultrasound transducers, magnetic resonance imaging apparatus, angiography apparatus, optical coherence tomography apparatus, and combinations thereof. Contemplated thermographers include, but are not limited to, thermocouples, thermosensors, thermistors, thermometers, spectrography devices, infrared thermographers, fiber optic infrared thermographers, ultrasound-based thermographers, spectroscopy devices, near infrared spectroscopy devices, and combinations thereof.

Contemplated stabilization elements include, but are not limited to, balloons, stents, coated stents, covered stents, stent grafts, eluting stents, drug-eluting stents, magnetic resonance stents, anastamosis devices, ablation devices, photonic ablation devices, laser ablation devices, RF ablation devices, ultrasound ablation devices, therapeutic ultrasound transducers, sonotherapy elements, coronary bypass devices, myocardial regeneration devices, sonotherapy devices, drug delivery devices, gene therapy devices, atherectomy devices, heating devices, plaque rupture devices, secondary-substance modifiers, therapeutic agents, contrast agents, drug capsules, tissue-type tags, extreme lipid lowering agents, cholesterol acyltransferase inhibitors, matrix metalloproteinase inhibitors, anti-inflammatory agents, anti-oxidants, angiotensin-converting enzyme inhibitors, radiation elements, brachytherapy elements, local drug injection elements, gene therapy elements, photodynamic therapy elements, photoangioplasty elements, cryotherapy elements, and combinations thereof. Additional imaging elements, thermographers, and optional stabilization elements will be apparent to those of skill in the art. The appended claims are intended to cover all combinations of imaging elements, thermographers, and, optionally, stabilization elements that fall within the true spirit and scope of the present invention.

Furthermore, apparatus of the present invention may optionally be provided with a distal protection device (not shown), in order to capture emboli and/or other material released, for example, during stabilization of vulnerable plaque. Distal protection devices are provided, for example, in U.S. Pat. No. 6,348,062 to Hopkins et al., and U.S. Pat. No. 6,295,989 to Connors, III, both of which are incorporated herein by reference. Additional distal protection devices, per se known, will be apparent to those of skill in the art. The appended claims are intended to cover all such changes and modifications that fall within the true spirit and scope of the invention.

What is claimed is:

1. Apparatus for identification of vulnerable plaque, the apparatus comprising:
a catheter having a distal region;
an imaging element disposed on the catheter in the distal region; and
a thermographer disposed on the catheter adjacent the imaging element.

2. The apparatus of claim 1, wherein the imaging element is chosen from the group consisting of ultrasound transducers, linear-array ultrasound transducers, phased-array ultrasound transducers, rotational ultrasound transducers, forward-looking ultrasound transducers, radially-looking ultrasound transducers, magnetic resonance imaging apparatus, angiography apparatus, optical coherence tomography apparatus, and combinations thereof.

3. The apparatus of claim 1, wherein the thermographer is chosen from the group consisting of thermocouples, thermosensors, thermistors, thermometers, spectrography devices, infrared thermographers, fiber optic infrared thermographers, ultrasound-based thermographers, spectroscopy devices, near infrared spectroscopy devices, and combinations thereof.

4. The apparatus of claim 1 further comprising a stabilization element.

5. The apparatus of claim 4, wherein the stabilization element is chosen from the group consisting of balloons, stents, coated stents, covered stents, stent grafts, eluting stents, drug-eluting stents, magnetic resonance stents, anastamosis devices, ablation devices, photonic ablation devices, laser ablation devices, RF ablation devices, ultrasound ablation devices, therapeutic ultrasound transducers, sonotheraphy elements, coronary bypass devices, myocardial regeneration devices, sonotherapy devices, drug delivery devices, gene therapy devices, atherectomy devices, heating devices, plaque rupture devices, secondary-substance modifiers, therapeutic agents, contrast agents, drug capsules, tissue-type tags, extreme lipid lowering agents, cholesterol acyltransferase inhibitors, matrix metalloproteinase inhibitors, anti-inflammatory agents, anti-oxidants, angiotensin-converting enzyme inhibitors, radiation elements, brachytherapy elements, local drug injection elements, gene therapy elements, photodynamic therapy elements, photoangioplasty elements, cryotherapy elements, and combinations thereof.

6. The apparatus of claim 4 further comprising a distal protection device.

7. The apparatus of claim 1, wherein the apparatus is adapted to perform a function chosen from the group consisting of elastography, palpography, and blood flow imaging.

8. The apparatus of claim 1 further comprising a graphical user interface for displaying imaging and thermography data obtained with the imaging element and thermographer, respectively.

9. The apparatus of claim 8, wherein the imaging and thermography data are coupled and displayed simultaneously.

10. The apparatus of claim 9, wherein the graphical user interface is adapted to display imaging and thermography data obtained along a cross-section of a patient's vessel.

11. The apparatus of claim 9, wherein the graphical user interface is adapted to display imaging and thermography data obtained along a side-section of a patient's vessel.

12. The apparatus of claim 1 further comprising position indication elements for determining the position of the imaging element and the thermographer.

13. The apparatus of claim 1 further comprising a pullback system coupled to the catheter.

14. The apparatus of claim 8, wherein the graphical user interface is further adapted to display data chosen from the group consisting of palpography data and blood flow data.

15. A method for identifying vulnerable plaque within a body lumen of a patient, the method comprising:
providing apparatus comprising a catheter having both an imaging element and a thermographer;
percutaneously advancing the catheter to a target region within the patient's body lumen;
obtaining an image of the target region with the imaging element;
obtaining temperature data at the target region with the thermographer; and
comparing the image and the temperature data obtained at the target region to determine if vulnerable plaque is present at the target region

16. The method of claim 15, wherein an increase in temperature at the target region is indicative of vulnerable plaque.

17. The method of claim 15, wherein eccentric stenosis observed in the image is indicative of vulnerable plaque.

18. The method of claim 15, wherein echolucent zones observed in the image are indicative of vulnerable plaque.

19. The method of claim 15, wherein comparing the image and the temperature data further comprises coupling and simultaneously displaying the image and the temperature data.

20. The method of claim 15 further comprising obtaining additional data at the target region with the imaging element,
wherein comparing the image and the temperature data further comprises comparing the image, temperature and additional data obtained at the target region to determine if vulnerable plaque is present at the target region.

21. The method of claim 20, wherein obtaining additional data comprises obtaining additional data chosen from the group consisting of palpography data and blood flow data.

22. The method of claim 15 further comprising stabilizing the target region at locations where vulnerable plaque has been identified.

23. The method of claim 22 further comprising providing distal protection while stabilizing the target region at locations where vulnerable plaque has been identified.

24. Apparatus for identification of vulnerable plaque, the apparatus comprising:
a catheter having proximal and distal ends, and a bore extending from the proximal end towards the distal end;
a rotatable drive cable having a distal region; and
a side-viewing fiber optic coupled to the distal region of the drive cable,
wherein the rotatable drive cable is disposed within the bore, and
wherein the side-viewing fiber optic is proximally coupled to an infrared thermography system.

25. The apparatus of claim 23, wherein the rotatable drive cable is coupled to a driver adapted to rotate the drive cable and side-viewing fiber optic, thereby providing the side-viewing fiber optic with a 360° field of view.

26. The apparatus of claim 23 further comprising an imaging element.

27. The apparatus of claim 25, wherein the imaging element comprises an ultrasound imaging transducer coupled to the distal region rotatable drive cable.

28. The apparatus of claim 26, wherein the ultrasound imaging transducer is proximally coupled to an ultrasound imaging system.

29. The apparatus of claim 23 further comprising a stabilization element.
